SEQUENCE LISTING (20 sequences)

SEQ ID NO: 1 - protein, 362 aa, Solanum lycopersicum
MGRAPCCDKNGLKKGPWTPEEDQKLIDYIQKHGYGNWRTLPKNAGLQRCGKSCRLRWTNYLRPDIKRGRFSFEEEETIIQLHSILGNKWSAIAARLPGRTDNEIKNYWNTHIRKRLLRMGIDPVTHSPRLDLLDLSSILNHSIYNNSSHHQMNLSRLLGHVQPLVNPELLRLATSLISSQRQNTNNFLIPNNLQENQIICQNQLPQMVQNNQIQDFSTISTTPCVPFSSHEAQLMQPPITTKIEDFSSDLENFGNSQNNCQVINDDEWQLSNGVTDDYFPLQNYGYYDPLTSDENNNNFNLQSVVLSNLSTPSSSPTPLNSNSTYFNNSSSTTTEDERDSYCSNMLNFDNIPNIWDTTNEFM

SEQ ID NO: 2 - protein, 313 aa, Solanum lycopersicum
MGRTPCCDKNGLKKGPWTTEEDQKLIDYIQKYGSGNWRILPKNAGLQRCGKSCRLRWINYLRPDIKRGKFSFEEEETIIHLHSILGNKWSAIAARLPGRTDNEIKNYWNTNIRKKLLRMGIDPITHSPRLDLLDLNSIFNPSLYNSTQLDNNISRLLGVQSLVNPEILRLANSLLSSHHQNQNFLLQSNFQENQLCNSYVQNKLTPFGQTSLIQNPINNISTCSNFNTPSVPFYSDTLAMQQPNVEEQSSSNILNFNSQNFTFNSILPTLSTPSSTPTSLNSNSSTISEEERESYCSMLNFDIPNILDVNEFM

SEQ ID NO: 3 - protein, 347 aa, Solanum lycopersicum
MGRSPCCDENGLKKGPWTPEEDQKLTNHINKHGHGSWRALPKLAGLNRCGKSCRLRWSNYLRPDIKRGKFSQEEEQTILNLHAVLGNKWSAIATHLPGRTDNEIKNFWNTHLKKKLIQMGYDPMTHRPRTDIFDSLQHLIALVNLKELIESHSWEEQAMRLHYLQNLLQQPHNNMSTLSGIQNVEAYNLLNSLGDSQFLSTNNNNLGNHIVQQIPSSLDQPIIQDSISFSHLPELHTPSSFQTSLNKDRVRTEDTEFRIMSQGETSPASPWLPSLSPPPPPQVMNDQRSKENSSEVVISSGLSGESKNSNHLFLPDNKQSLNNIEEAPPSIWSDLLEDSFFQDIDKF

SEQ ID NO: 4 - protein, 243 aa, Solanum lycopersicum
MGKGRTACCDKSKVKKGPWTPSEDLKLISFIQKHGHGNWRALPKQAGLLRCGKSCRLRWINYLRPDVKRGNFTPQEEDTIINLHRAFGNRWSKIASHLPGRTDNEIKNVWNTHLKKRLVVMKKEECKSSSSSTSTSSHQGQYMDNNNNNNSNTLESFSPTSSKANDQVMDFWEYMLDTSSTTTSINNLDHLDSYSKLDITSEHHPQQLVDEYECQKWLTYLEIELGLTTNNQQEDHQNNFMQL

SEQ ID NO: 5 - protein, 417 aa, Solanum lycopersicum
MGRSPCCDKVGLKKGPWTPEEDQKLLAYIEEHGHGSWRALPTKAGLQRCGKSCRLRWTNYLRPDIKRGKFTLQEEQTIIQLHALLGNRWSAIATHLSKRTDNEIKNYWNTHLKKRLVKMGIDPVTHKPKNDALLSNDGQSKNAANLSHMAQWESARLEAEARLARQSKLRSNSFQNSLASQEFTAPSPSSPLSKPVVAPARCLNVLKAWNGVWTKPMNEGSVASASAGISVAGALARDLESPTSTLGYFENAQHITSSGIGGSSNTVLYEFVGNSSGSSEGGIMNNDESEEDWKEFGNSSTGHLPQYSKDVINENSISFTSGLQDLTLPMDTTWTTESSRSNTEQISPANFVETFTDLLLSNSGDGDLSEGGGTESDNGGEGSGSGNPNENSEDNKNYWNSIFNLVNNPSPSDSSMF

SEQ ID NO: 6 - protein, 253 aa, Solanum lycopersicum
MPRVQQQQQKGTSMEAIIKKGAWSPEEDQKLRGYIMKYGIWNWRQMPKFAGLSRTGKSCRLRWMNYLRPDVKRGPFTTEEVEIVIKTYQELGNSWSAIAAKLPGRTDNEVKNFFHTHLKKHLGLKNHDVPLKTRKIRKQTKEDEKKISTRGRLVLETSNNSNLLTTDVCSPCSSITTCEENQMMDPFVNFSQTFEVCYNNITSLVVDQQVPGMEHTCINIGVAQPHSIPHGPAVNSFDQFDMNSFWIDVLGNI

SEQ ID NO: 7 - protein, 313 aa, Solanum lycopersicum
FSKISSQEKKIIIIIIIMGRAPCCDKANVKKGPWSPEEDAKLKEYIDKFGTGGNWIALPQKAGLRRCGKSCRLRWLNYLRPNIKHGEFSDEEDRIICSLYANIGSRWSIIAAQLPGRTDNDIKNYWNTKLKKKLMGFVSSSHKIRPLNHHDYHHQIPTNCYNNYSSLVQASSLLISSNYPNNTTFPCYETNIPSTTPSSTSFLSAGASTSCTSGITASTFAGRTTSSDESYDISNFNFHSYMYNNNGVISEGEKLISGNNASGCYVDEQQNPLDYSSLEEIKDLISTNHGTCNSTSFLLDHEIKTEEKVIMYY

SEQ ID NO: 8 - protein, 699 aa, Solanum lycopersicum
MYYQGTSDNNIQADHHQQQHNNLGNSNNNIQTLYLMNPNSYMQGYTTTDTQQHLQQQQNQHQLLFLNSAPAGGNALSHANIQHAPLQQQHFVGVPLPAVSLHDQINHHGLLQRMWNNQDQSQQVIVPSSTVVSATSCGGTTTDLASQLAFQRPIVVSPTPQHRQQQQQQGGLSLSLSPQQQQQISFNNNISSSSPRTNNVTIRGTMDGCSSNMILGSKYLKAAQELLDEVVNIVGKSNKGDDQKKDNSMNKELIPLVSDVNTNSSGGGGGESSSRQKNEVAIELTTAQRQELQMKKAKLLAMLEEVEQRYRQYHHQMQIIVSSFEQVAGVGSAKSYTQLALHAISKQFRCLKDAISEQVKATSKSLGEDEGLGGKIEGSRLKFVDHHLRQQRALQQLGMMQPNAWRPQRGLPERAVSVLRAWLFEHFLHPYPKDSDKIMLAKQTGLTRSQVSNWFINARVRLWKPMVEEMYLEEVKNQEQNSSNTSGDNKNKETNISAPNEEKQPIITSSLLQDGTTQAEISTSTISTSPTAGASLHHAHNFSFLGSFNMENTTTTVDHIENNAKKPRNHDMHKFSPSSILSSVEMEAKARESTNKGFTNPLMAAYAMGDFGRFDPHDQQMTANFHGNNGVSLTLGLPPSENLAMPVSQQNYLSNELGSRPEIGSHYNRMGYENIDFQSGNKRFPTQLLPDFVTGNLGT

SEQ ID NO: 9 - protein, 438 aa, Solanum lycopersicum
MDDIPEWLKGLPLAPEFRPTDTEFADPIAYISKIEKEASAFGICKVIPPLPKPSKKYVLHNLNNSLSKCPDLNSAGAPVFTTRHQELGHTEKKKFPFGAQKQVWQSGQLYTLDQFETKSKNFARTQFGIVKDISPFLVEAMFWKTAFDHPIYVEYANDVPGSAFGEPEENFCRTKRPRNRKILDRTSSTTSVDKGRSHHSVDTPSSSLLTPLSNSSPFRPKGCSNAAEMEGSAGWKLANSPWNLQVIARSPGSLTRFMPDDIPGVTSPMVYIGMLFSWFAWHVEDHELHSLNFLHTGSPKTWYAVPGDYAFSFEEVIRCHAYGETTDRLVNLGHKALKFASGKAKATYTEQHEDFVVRLCTSNHEIGCLGEQAALALLGEKTTLLSPEVLVASGIPCCRLVQNPGEFVVTFPRAYHVGFSHETEIFIGLRTYDSLWRS

SEQ ID NO: 10 - protein, 217 aa, Solanum lycopersicum
MSNSPVFEPLGTSVYLRQRDLLQKFCQENIANISIPTTSKTIPFRNSLYTQSYKLPEKKKLYRGVRQRHWGKWVAEIRLPQNRMRVWLGTYETAEAAAYAYDRAAYKLRGEYARLNFPNVRDPSKLGFGDGEKMNAVKNAVDAKIQAICQRVKREKAKKAAKKKSENENGLWRSEDSTCSVFGDCLKDPLMESEFDSCSLARMPSFDPELIWEVLAN

SEQ ID NO: 11 - protein, 298 aa, Solanum lycopersicum
MALETVVFQQDPFNYSHKDCNFYNLETFHDYGNFGYEGYNWNSSIPQSYNDDDNNNNINNNNSNSSPDKYFPVESTVVSGRRKRRRTKCAKNEEEIHNQRMTHIAVERNRRRQMNDYLAVLRSLMPPSYAQRGDQASIVGGAINFVKELEQLLQFLEAHKQVITTNQQHIQYSSFSKFFTFPQYSTGNNNHPLAATTSNEGSEERRSAVADIEVTMVESHANVKVLSRRRPKQLLKIVNWLQAMCLTILHLSVTTADHMVLYTFSVKVEENCELNTVSEIASAVHEMVAMIKEEAMPC

SEQ ID NO: 12 - protein, 424 aa, Solanum lycopersicum
MSNSNLSSGNSSEEADETPYVLSSTSDGSSAHQQHSQTNNKKRRKLPGNPDPSAEVIALSPKTLMATNRFICEVCNKGFQREQNLQLHRRGHNLPWKLKQKTSNEIKKRVYICPESSCIHHNPSRALGDLTGIKKHFSRKHGEKKWKCEKCSKKYAVQSDWKAHSKTCGTKEYKCDCGTIFSRRDSFVTHRAFCDALAEENNKVNQVLASTTQPLATGPELISTTQMLNLPQIRNSNMKIPSIPLNMAGSMFSSSSGFNQLGTNSSNMSSATALLQQAAQMGATVNNNMNSTLFNGVQIPIQSNHDHDQNETQIGSILQGFGGSMLQNNGDDHHKSSRVLQNEQGWFNNNNNNSNTGLFNEKQRTLNKEAGHSNEESLTLDFLGIGGMRHRNLHEMHQHQQEMSFEQQQVNHQSIQRVNSIWDD

SEQ ID NO: 13 - protein, 433 aa, Solanum lycopersicum
MENGKHSVAIELTVKQGVPSLVSPAEETEKGPYYLSNLDQNIAVPVRTIYCFKSEEKGNDNAAEVMKDALSKVLVHYFPLAGRLTISQEMKLIVDCSGEGAVFVEAEANCNIEDIGDNTKPDPVTLGKLVYDIPGAKNILEMPPLVAQVTKFKCGGFVLGLCMNHCMFDGIGAMEFVNSWGEIARGLPIKVPPFLDRSILKPRNPPKPEYTHNEFAEIKDISDSTKLYQEEMMYKAFCFDPEKLEQLKAKAKEDGNVTKCTSFEVLSAFIWKARTQALQMKPDQKTKLLFAVDGRSRFDPSIPRGYFGNGIVLTNALCTAAEIVENPLSVAVKLVQEAVKLVTDSYMKSAIDYFETTRARPSLTATLLITTWSRLSFHTTDFGWGEPIVSGPVALPEKEVSLFLSHGKERRSVNVLLGLPASAMKTFEELMEI

SEQ ID NO: 14 - protein, 491 aa, Solanum lycopersicum
MDSSIVCELEGTLLKDQDPFSYFMLIAFEASSLIRFAILLMLWPLIKFLGICGQKDKGLKLMIFVATIGVKISEIEVVARAVLPKFYFDDIDMKSWRIFSSFDKRIVVTKIPRIMVERFVKEHLRADDVIGSELVVNNFGFATGFIKDDFDSILERVGALFDGETQPSLGLGRPQNGSSFLSLCKEQLHPPFMINKNQDHIIKPLPVIFHDGRLVKRPTPSIALLILLWIPFGIILATIRIIIGLILPLWIVPYLAPLFGGKVIVKGKPPPPASITNSGVLFVCTHRTLLDPVVLSTVLQRRIPAVTYSISRLSEILSPIPTVRLTRIREVDAQKIKRQLEKGDLVVCPEGTTCREPFLLRFSALFAELTDRIVPVAMNYRVGFFHATTARGWKGMDPIFFFMNPRPMYEVTFLNQLPVEATCSSGKSPHDVANYVQRILAATLGFECTNFTRKDKYRVLAGNDGIVSQNSGTNLANKFKKVVATFKLFIH

SEQ ID NO: 15 - protein, 501 aa, Solanum lycopersicum
MSLPKSKKSFPSVTTCDTSAVNHHSVAADLDGTLLISRSSFPYFMLVAIEAGSLFRGLILLLSFPLIAIAYVFVSEALAIQMLIYISFAGLKVRDIELASRAVLPRFYATDVRKESFEVFDQCKRKVVVTANPTIMVEPFVKDFLGGDKVLGTEIEVNPKTKKATGFVKSPGILVGKWKKLSILKEFGEEMPDIGLGDRESDHDFMSICKEGYMVLPSESAKPVPLDRLKSRLIFHDGRLVQRPTPFNALVTYIWLPFGFALGVFRVYFNLPLPERIVRYTYGMVGINLVIKGPRPPPPSPGTPGNLYVCNHRSALDPIVIAIALGRKVSTVTYSVSKLSRFLSPIPAIALTRDREADAAMIKKLLEKGDLVVCPEGTTCREPFLLRFSALFAELSDRIVPVAVDTKQSMFFGTTVRGVKFWDPYFFFMNPRPTYELTFLEPLPMEMTCKAGKTSIEVANHVQKVLGGVLGFECTQLTRKDKYMLLGGNDGKVESMYSKKA

SEQ ID NO: 16 - protein, 673 aa, Solanum lycopersicum
MEDQKKLYVFEVEKAKEVRSNGRPSRGPVYRNVLAKDGFRPLSQSLQSCWDIFCESVRKFPHNRMLGEREMSHGQAGKYIWLTYREVYDLVLKVGASMRVCGVKQVRLSYCKIKDIQGGKCGIYGANCSNWVISMQACNALGLYCVPLYDTLGAGAVEYIICHAEVSVAFAEETKIFEVLKAFPNAGKFLKSLISFGKVTQEQKDMAGNFDLKLYSWDEFLLLGMQEKFDLPAKKKTDICTIMYTSGTTGDPKGVMISNESILSLISGVNHHMETVGEEFTDKDVYLSYLPLAHIFDRVIEELFISKGASVGFWHKDVKQLIDDIKELKPTVFCSVPRVLDKIYSGLVEKISCAGFLKHKLFNFTYNYKLGNMSKGYRHSEAAPIFDKIIFNKVKEGLGGNLRLILSGAAPLSSTVETYLRVVTCANVLQGYGLTETCAGSFVARPDELAMVGTVGPPLPIIDVCLESVPEMGYDALGDTPRGEICIRGKCLFSGYYKREDLTKEVLVDGWFHTGDVGEWQPDGSMKIVDRKKNIFKLSQGEYVAVENLEGIYSLASSVDSIWIYGSSYESFLVAVVNPNMEALRSWANENGMTGDFDTICENPKAKAYILSELTNIAKEKKLKGFEFIKAVHLDPVPFDMERELITPTHKKKRAQFLKYYQNNIDTLYKNTR

SEQ ID NO: 17 - protein, 526 aa, Solanum lycopersicum
MDIAIALLLFSFITCYLLWFTFISRSLKGPRVWPLLGSLPGLIENSERMHEWIVDNLRACGGTYQTCICAIPFLARKQGLVTVTCDPKNLEHILKTRFENYPKGPTWQAVFHELLGQGIFNSDGDTWLFQRKTAALEFTTRTLRQAMARWVNRAIQLRFCPILKTAQVEGKPVDLQDLLLRLTFDNICGLAFGKDPQTLAPGLPDNTFASAFDRATEASLQRFILPEVVWKLKKWLGLGMEVSLNRSLVQLDKYMSDIINTRKLELMSQQKDGNPHDDLLSRFMKKKESYTDKFLQHVALNFILAGRDTSSVALSWFFWLVIQNPVVEQKILQEISTVLVETRGSDTSSWLEEPLAFEEVDRLTYLKAALSETLRLYPSVPEDSKHVVVDDVLPDGTFVPAGSSITYSIYSAGRMKSTWGEDCLEFKPERWLTLDGKKFVMHEQYKFVAFNAGPRICLGKDLAYLQMKSVAAAVLLRHRLTVAPGHKVEQKMSLTLFMKDGLKVNLRPRELTPFVNSVKEVQLIQI

SEQ ID NO: 18 - protein, 491 aa, Solanum lycopersicum
MELTSVLKFLENRAILVTGATGFLAKIFVEKILRVQPNVKKLYLLLRAQDNNAALQRFNNEAVAKDLFKLLREKHGANLNTFISERTTIIPGDITIENLGVKDTNLLEEMWREVDVVVNLAATTNFDERYDVALGLNTFGAINVLNFAKKCSKLKVLLHVSTAYVSGEKRGLILETPYNLGETLNGTSGLDIYTEKKVMEETLKQLRVEGSSQESITSAMKELGLQRARKYGWPNPYVFTKALAEMILGDMKEDVLLVIFRPTIVTSTLRDPFPGWVEGIRTIDSLAVGYGKGKLTCFLGDPEAIIDLIPADMVVNAMIVTMMAHADQRGSQIIYHVGTSVSNPVKFTCPQEYAFRHFKEHPWIDKQGKPVIVGKVNVLSSMDSFRRYMALRYMLPLKGLEIVNTILCQFFQDKYSELDRKIKFVMRLIDLYEPYLFFKGVYDDMNTEKLRRAAKESGIETDVFNFNPKSINWEDYFMNTHIPGVVKYVFK

SEQ ID NO: 19 - protein, 492 aa, Solanum lycopersicum
MEMTSVLNFLENRTILVTGATGFLAKIFVEKILRVQPYVKKLYLLLRAADDKSAMQRFNTEVVGKDLFKVLREKCGPNFTTFVSQRTTIVPGDITCENLGVNDTNLLEQMWKEVDIVVNLAATTNFDERYDVALGLNTFGASHVLNFAKKCNKLKVLLHVSTAYVCGEKEGLMLEKPYYMGETLNGTLGLDIEAEKKVMDEKLKQLKAENASEKSITTAMKELGLERARKYGWPNTYVFTKAMGEMLLGKLKEEVPLVINRPTIITSTFKEPFPGWVEGIRTIDSLAVGYGKGRITCFLGNPKTILDVIPADMVVNSMIVAMMAHADQKGSETIYQIGSSVSNPLNITNLRDYGFNYFRKNPWINKVNGKPIIVGKVNVLSSMDSFQRYMALHYILPLKGLEIVNAAFCQYFQGKYLELYKKIKFVMRLIDLYGPYLFLKAAFDDLNTEKLRIGAKESGIETEIFYFDPKIINWEDYFMKIHLPGVVRYVFK

SEQ ID NO: 20 - protein, 513 aa, Solanum lycopersicum
MGDESTRRVSIEANSNKLPNFLLSVRLKYVKLGYHYLISHAMYLFLIPILMALFAHLSTITMEDMVQLWNQLKFNLVTVILCSALIVFLATLYFMTRPRKVYLVDFSCYKPKPEVMCPKELFMERSKLAGIFTEENLAFQKKILERSGLGQKTYFPEALLKLPPNPCMAEARKEAEMVMFGAIDELLEKTGVKAKDIGILVVNCSLFNPTPSLSAMIVNHYKLRGNILSYNLGGMGCSAGLISIDLAKQMLQVQPNSYALVVSMENITLNWYFGNNRSMLVSNCIFRMGGAAILLSNKSSDRKRSKYQLIHTVRTHKGADDKSYGCVFQEEDDNKKIGVALSKDLMAVAGEALKTNITTLGPIVLPMSEQLLFFATLVARKVLKMKIKPYIPDFKLAFEHFCIHAGGRAVLDELEKNLELSEWHMEPSRMTLYRFGNTSSSSLWYELAYTEAKGRIKKGDRTWQIAFGSGFKCNSAVWCALRTINPAKEKNPWMDEIDEFPVEVPRVVTINDS

(19) United States
(12) Patent Application Publication
(10) Pub. No.: US 2023/0175002 A1
(43) Pub. Date: Jun. 8, 2023

(54) SUBERIN BIOSYNTHETIC GENES AND REGULATORS

(71) Applicant: The Regents of the University of California, Oakland, CA (US)

(72) Inventors: Siobhan M. BRADY, Davis, CA (US); Alex CANTO-PASTOR, Davis, CA (US); Mona GOURAN, Davis, CA (US); Kaisa KAJALA, Utrecht (NL); Dorota KAWA, Davis, CA (US); Grace Alex MASON, Davis, CA (US); Concepcion MANZANO, Davis, CA (US); Niba NIRMAL, Durham, NC (US); Lidor SHAAR-MOSHE, Davis, CA (US); Julie BAILEY-SERRES, Riverside, CA (US); Alex BOROWSKY, Riverside, CA (US); Mauricio REYNOSO, Riverside, CA (US); Neelima SINHA, Davis, CA (US)

(21) Appl. No.: 17/916,516

(22) PCT Filed: Apr. 2, 2021
(86) PCT No.: PCT/US21/25548

§ 371 Date: Sep. 30, 2022

Related U.S. Application Data
(60) Provisional application No. 63/005,036, filed on Apr. 3, 2020.

(51) Int. Cl. C12N 15/82

(52) U.S. Cl. CPC C12N 15/8243; C12N 15/8218; C12N 15/8273

(57) ABSTRACT

The present disclosure provides a list of genes, and the proteins encoded by these genes, that modulate and/or participate in the synthesis of the biopolymer suberin. The genes described here are useful in methods for producing genetically modified plants or breeding plants with altered production (enhanced or disrupted) of suberin. Such plants can contain modified or mutated candidate peptides; or have disrupted expression of using methods such as clustered regularly-interspaced short palindromic repeats (CRISPR)/CRISPR associated (Cas) nuclease, an antisense nucleic acid, a zinc finger nuclease (ZFN), or a transcription activator-like effector (TALE) nuclease. Suberin has a positive influence on response to plant water stress, a long-lasting role as a carbon sink in soil; and the lack of suberin encourages symbioses for nutrient uptake as well as for prevention of pathogenicity.

### CROSS-REFERENCE TO RELATED APPLICATIONS

The present application claims benefit of priority to U.S. Provisional Patent Application No. 63/005,036, filed Apr. 3, 2020, which is incorporated by reference for all purposes.

### STATEMENT AS TO RIGHTS TO INVENTIONS MADE UNDER FEDERALLY SPONSORED RESEARCH AND DEVELOPMENT

This invention was made with Government support under Grant No. #IOS-123824, awarded by the National Science Foundation. The Government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 081906-1238726-238210PC_SL.txt, created on Apr. 1, 2021, 73,567 bytes, machine format IBM-PC, MS-Windows operating system, is hereby incorporated by reference in its entirety for all purposes.

### BACKGROUND OF THE INVENTION

Suberin is a natural complex carbon-rich biopolymer typically found in cell walls of plants. Suberized cell walls form physiologically relevant interfaces between the plant and the environment: they act as barriers that limit water and nutrient transport and protect plants from invasion by pathogens. Suberin is present ubiquitously in specific internal root-tissues of vascular plants, which suggests that this polymer played an important role in the adaptation of plants to terrestrial life.

Plants respond to external stimuli by modifying the abundance of suberin in their root cell walls. In the case of drought, salt stress or oxygen deficiency, suberization is increased in roots. However, the genetics determining suberin deposition and regulation in most plant species remain largely unknown.

There is increasing incentive in agriculture to develop cultivars with enhanced tolerance to stresses, from drought to pests.

Evidence that Up-Regulation of Suberin Leads to Drought Tolerance

Baxter et al. PLoS Genetics (2009): The Arabidopsis esb1 mutant, characterized by increased root suberin, was found to have reduced transpiration and increased water-use efficiency. Baxter et al. found evidence that suberin in the roots plays a role in controlling both water and mineral ion uptake and transport to the leaves. They also showed that esb1 roots had increased resistance to drought.

Serra, O. et al. (2009), Plant Physiology, 149(2), 1050-1060: This work generated potato plants with reduced suberin through RNAi silencing of CYP86A33 (a gene involved in suberin biosynthesis). The water permeability of the periderm of CYP86A33-silenced plants was 3.5 times higher than that of the wild type; thus providing clear evidence that aliphatic suberin is relevant for the water permeability and drought.

Evidence that Suberin Will Increase Carbon Sequestration

Poirier V., Roumet C., Munson A. D. (2018), Soil Biology and Biochemistry, 120: 246-259: Suberin promotes soil organic matter stabilization in both short (1-10 yrs), intermediate (10-100 yrs) and passive (>100 yrs) pools through three different aspects: selective preservation through recalcitrance, stabilization through macro aggregation and interaction with minerals and metals. In other words, increased suberin will stabilize more soil organic matter, which means more carbon sequestration in the soil.

Evidence that Suberin Levels Correlate with Pathogen Tolerance

Thomas, R. et al. (2007), Plant Physiology, 144(1), 299-311: This paper showed that significantly higher amounts of suberin in tissues isolated from a soybean cultivar (‘Conrad’) positively correlated with resistance to the oomycete Phytophthora sojae, compared with a susceptible line (OX760-6). This correlation was extended by an analysis of nine independent and 32 recombinant inbred lines (derived from a ‘Conrad’ 3 OX760-6 cross) ranging in resistance to P. sojae. Both aliphatic and phenolic suberin levels proved to be correlated with resistance to the oomycete. This meant that susceptibility to P. sojae decreases with increasing amounts of suberin.

Holbein, J. et al. (2019), The Plant Journal, 100(2), 221-236: This work showed that nematode infection damages the Arabidopsis endodermis leading to the activation of suberin biosynthesis genes at nematode infection sites. Using endodermal barrier-deficient mutants (a defective Casparian strip without suberin), they also showed that lack of suberin renders the plant more susceptible to nematode parasitism, particularly for the root-knot nematode Meloidogyne incognita.

Evidence that Over-Expression of Transcriptional Regulators of Suberin Biosynthesis Will Lead to Increased Suberin.

Kosma D. K et al. (2014), Plant J, 80: 216-229: It has been shown for one Arabidopsis transcription factor, AtMYB41, that its overexpression is able to drive biosynthesis and deposition of suberin-like lamellae in tissues that do not usually accumulate suberin (leaf epidermis and mesophyll) in multiple species (Arabidopsis, Nicotiana benthamiana).

Cohen et al. Plant J. (Feb. 6, 2020): SUBERMAN, an Arabidopsis transcription factor involved in the deposition of suberin in the roots, was ectopically expressed in Nicotiana leaves. This transient expression resulted in the induction of heterologous suberin genes, the accumulation of suberin-type monomers, and consequent deposition of suberin-like lamellae. The overall results suggest a high conservation of suberin deposition pathways across plant species. Furthermore, it reinforces the validity of an approach based on transgenic expression of transcription factors ectopically and/or cross-species.

Evidence that Increased Suberin Leads to Increased Shelf Life

Landgraf, R. et al. (2014), The Plant Cell, 26(8), 3403-3415: the potato ABCG1 transporter, involved in suberin formation in the root and periderm, was silenced. Transgenic ABCG1-RNAi potato display major alterations in both root and tuber morphology. In accordance with the reduced suberization of the periderm, ABCG1-RNAi tubers suffered a severe water loss during 20 d of storage, resulting in a 2-fold weight reduction, whereas control tubers did not lose weight to a significant extent.

Serra, O. et al. (2010), The Plant Journal, 62(2), 277-290: Similar to potato ABCG1, this work showed that silencing of potato FTH (homolog to tomato ASFT) had significant effects on cell anatomy, sealing properties and maturation of the periderm. The tuber skin became thicker and russeted, water loss was greatly increased, and maturation was prevented.

Evidence that Increased Suberin Leads to Increased Salinity, Waterlogging and Drought Tolerance

The following pieces support the role of suberin for abiotic stress tolerances:

Salinity: (Krishnamurthy, P. et al. (2009), Planta, 230, 119-134): The increasing root suberin content negatively correlates with the accumulation and transport of sodium into shoots in rice, protecting the root against overaccumulation of salt.

Waterlogging (Kotula, L. et al. (2009), J. Exp. Bot., 60, 2155-2167): The increasing exodermal suberin content along the root axis correlates with decreasing radial oxygen loss in rice, protecting the root against loss of oxygen into the hypoxic waterlogged soil.

Drought: (Taleisnik E. et al. (1998), Annals of Botany 83:19-27): The relative water retention ability is higher in the roots with exodermis.

### BRIEF SUMMARY OF THE INVENTION

Alteration of suberized cell wall composition would be a suitable option to improve plant stress tolerance. Since most crop products generally contain less suberin that their stress tolerant wild relative, a method for controlling suberin deposition would be economically valuable.

In some embodiments, the disclosure provides a plant having increased suberin, wherein the plant ectopically expresses or overexpresses one or more polypeptide that is substantially identical to one or more protein as provided in Table 1 or SEQ ID NOS: 1-20, wherein the plant has increased suberin compared to a control plant not ectopically expressing or overexpressing the one or more polypeptide. In some embodiments, the plant is a Solanaceous plant. In some embodiments, the plant comprises an expression cassette comprising a promoter operably linked to a polynucleotide encoding one of the polypeptides of Table 1 or SEQ ID NOS: 1-20. In some embodiments, the promoter is inducible or tissue-specific.

In some embodiments, the disclosure provides a tuber from the plant as described above or elsewhere herein.

In some embodiments, the disclosure provides a method of making suberin. In some embodiments, the method comprises providing the plant or tuber as described above or elsewhere herein; and extracting suberin from the plant or a part of the plant.

In some embodiments, the disclosure provides a method of cultivating plants that are tolerant to drought or high salinity conditions, the method comprising, cultivating the plant as described above or elsewhere herein under high salinity or drought conditions.

In some embodiments, the disclosure provides a plant having decreased suberin, wherein the plant is (a) mutated to reduce or knockout expression, or (b) expresses an siRNA or antisense polynucleotide to reduce expression, of one or more polypeptide that is substantially identical to one or more protein as provided in Table 1 or SEQ ID NOS: 1-20, wherein the plant has decreased suberin compared to a control plant that expresses the one or more polypeptide. In some embodiments, the plant is a Solanaceous plant.

Other aspects of the invention are disclosed elsewhere herein.

### BRIEF DESCRIPTION OF THE DRAWINGS

FIG. 1 shows data demonstrating reduction of suberin in GPAT5, ASFT and MYB92 deletion alleles. GPAT5—Solyc04g011600; ASFT—Solyc03g097500; MYB92—Solyc05g051550.

### DEFINITIONS

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantial identity” or “substantially identical,” used in the context of two nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using the methods described herein; preferably BLAST using standard parameters, as described below. Embodiments of the present invention provide for nucleic acids encoding polypeptides (and a heterologous promoter operably linked to a polynucleotide encoding the polypeptides) that are substantially identical to any of the proteins in Table 1 or any one of SEQ ID NOS: 1-20.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see. e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only substantially identical to a sequence of the gene from which it was derived. As explained herein, these substantially identical variants are specifically covered by reference to a specific nucleic acid sequence.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A “control plant” refers to a plant that can be compared to a plant as described herein to indicate the effect of a mutation or expression of a protein as described herein. An exemplary control plant is a plant that is otherwise identical or substantially identical to a test plant but that lacks the mutation or heterologously-expressed polypeptide or polynucleotide.

### DETAILED DESCRIPTION OF THE INVENTION

The inventors have identified a number of genes, and their gene products, that influence plant production of suberin. Accordingly, one or more of the gene products described herein can be overexpressed or ectopically expressed in a plant to result in increased suberin in the plant as a whole or in cells or tissues in which the gene products are expressed. Alternatively, one or more of the described genes can be mutated to reduce expression or activity, or eliminate production of, their encoded gene products, thereby reducing suberin production in plant cells or tissues in which the genes have been mutated.

Alternatively, expression of the gene products can otherwise be reduced, for example antisense or sense suppression of the gene products.

Upregulation (e.g., overexpression or ectopic expression) can result in a variety of beneficial phenotypes.

1. Upregulation of transcription factors and enzymes (any and collectively) identified herein will enhance the expression of suberin biosynthetic genes and, as a consequence, boost the production of suberin and associated molecules in the plant. Upregulation will lead to an improved tolerance of the plant to drought and salt concentration in the soil. It will also increase the resistance to plant pathogens. Greater suberin deposition will also lead to greater concentration of carbon in the roots inside the soil, allowing for increased carbon sequestration from the atmosphere into the soil.

2. Ectopic expression of transcription factors and enzymes (any and collectively) identified herein in alternative tissues (for example, but not limited to, tubers, fruits and seeds) will enhance levels of suberin in these tissues, or in specific cell types, for example, but not limited to, exodermis. An increased level of suberin in these will lead to reduced water loss, increased resistance to rotting, and increased shelf life of derived agronomical products, including but not limited to tubers such as potatoes.

3. Upregulation of any one or a combination of transcription factors and enzymes (any and collectively) identified herein will enhance the accumulation of suberin and its monomers.

This will provide a low-cost and renewable source for these components, which could later be efficiently extracted, for example but not limited to by chemical methods, and can used in industrial applications. These applications can include, but are not limited to, synthesis of hybrid co-polymers, resins, or fibers. Suberin extracts have also shown medical properties as cancer-preventing anti-mutagenic agents and as a firming anti-wrinkle agent in human skin.

Downregulation of suberin can result in a variety of beneficial phenotypes.

1. Disruption of suberin (for example but not limited to mutation of any one or a combination of the genes described herein will lead to partial or total loss of suberin in the plant. The loss of suberin in the root will lead to increased levels of colonization of the plant by beneficial microbes and greater beneficial interaction between plant and soil microbiome.

2. Loss of suberin in the root will alter the morphological and physical properties of the root. These changes can be applied to change the properties of certain roots and tubers, making them more appealing/suitable to human consumption.

Accordingly, the disclosure provides methods of modulating (increase or decrease) suberin levels in a plant by altering expression or activity of a protein substantially identical to one listed in Table 1 of from SEQ ID NOS: 1-20, for example, by introducing into a plant a recombinant expression cassette comprising a regulatory element (e.g., a promoter) operably linked to a polynucleotide encoding the protein.

In some embodiments, the disclosure provides for increasing and/or ectopically expressing one or more of the proteins in a plant. Such embodiments are useful as described above. In some embodiments, selective promoters are used to drive expression as discussed further below. Where enhanced expression of a gene is desired, the desired gene (or at least the polynucleotide encoding the protein) from the same species or a different species (or substantially identical to the gene or polynucleotide encoding the protein from another species) may be used. In some embodiments, to decrease potential sense suppression effects, a polynucleotide from a different species (or substantially identical to the gene or polynucleotide from another species) may be used.

Any of a number of means well known in the art can be used to increase expression or activity in plants. Any organ or plant part can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat), fruit, abscission zone, etc. Alternatively, one or several genes can be expressed constitutively (e.g., using the CaMV 35S promoter or other constitutive promoter).

One of skill will recognize that the polypeptides, like other proteins, can have different domains which perform different functions. Thus, the overexpressed or ectopically expressed polynucleotide sequences need not be full length, so long as the desired functional domain of the protein is expressed. Alternatively, or in addition, active proteins can be expressed as fusions, without necessarily significantly altering activity. Examples of fusion partners include, but are not limited to, poly-His or other tag sequences.

Alternatively, expression or activity of the proteins described herein can be reduced or inhibited. Any one or more of the genes provided in Table 1 can be knocked out or mutated to reduce suberin production in a plant or plant cell. For example, in some embodiments, the native gene sequence mutated or knocked out in a plant encodes a polypeptide identical or substantially identical (e.g., at least 70, 75, 80, 85, 90, or 95% identical) to a protein of Table I or of any one of SEQ ID NO: 1-20. Gene sequences can be readily identified in many plant species in view of known genome sequences and the conserved nature of the proteins.

In some embodiments, the gene sequence is knocked out in the plant. “Knocked out” means that the plant does not make the particular protein encoded by the gene. Knockouts can be achieved in a variety of ways. For the purposes of this document, a knock out can be achieved by a deletion of all or a substantial part (e.g., majority) or the coding sequence for a polypeptide identical or substantially identical to a protein of Table I or any one of SEQ ID NO: 1-20. Alternatively a knock out can be achieved by introduction of a mutation that prevents translation or transcription (e.g., a mutation that introduces a stop codon early in the coding sequence or that disrupts transcription). A knock out can also be achieved by silencing or other suppression methods, e.g., such that the plant expresses substantially less of the native protein (e.g., less than 50, 25, 10, 5, or 1% of native expression).

In some embodiments, the mutation introduced into the protein is a single amino acid change that reduces or eliminates the protein's activity. Alternatively, the mutation can include any number (e.g., 1, 2, 3, 4, 5, 6, 7, 8, 9, or 10 or more) of amino acid changes, deletions or insertions that reduce or eliminate the protein activity.

Methods for introducing genetic mutations into plant genes and selecting plants with desired traits are well known and can be used to introduce mutations or to knock out a protein. For instance, seeds or other plant material can be treated with a mutagenic insertional polynucleotide (e.g., transposon, T-DNA, etc.) or chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. Plants having mutated protein can then be identified, for example, by phenotype or by molecular techniques.

Modified protein chains can also be readily designed utilizing various recombinant DNA techniques well known to those skilled in the art and described for instance, in Sambrook et al., supra. Hydroxylamine can also be used to introduce single base mutations into the coding region of the gene (Sikorski et al., Meth. Enzymol., 194:302-318 (1991)). For example, the chains can vary from the naturally occurring sequence at the primary structure level by amino acid substitutions, additions, deletions, and the like. These modifications can be used in a number of combinations to produce the final modified protein chain.

Alternatively, homologous recombination can be used to induce targeted gene modifications or knockouts by specifically targeting the gene in vivo (see, generally, Grewal and Klar, Genetics, 146:1221-1238 (1997) and Xu et al., Genes Dev., 10:2411-2422 (1996)). Homologous recombination has been demonstrated in plants (Puchta et al., Experientia, 50:277-284 (1994); Swoboda et al., EMBO J., 13:484-489 (1994); Offringa et al., Proc. Natl. Acad. Sci. USA, 90:7346-7350 (1993); and Kempin et al., Nature, 389:802-803 (1997)).

In applying homologous recombination technology to a gene, mutations in selected portions of gene sequences (including 5′ upstream, 3′ downstream, and intragenic regions) can be made in vitro and then introduced into the desired plant using standard techniques. Since the efficiency of homologous recombination is known to be dependent on the vectors used, use of dicistronic gene targeting vectors as described by Mountford et al., Proc. Natl. Acad. Sci. USA, 91:4303-4307 (1994); and Vaulont et al., Transgenic Res., 4:247-255 (1995) are conveniently used to increase the efficiency of selecting for altered PP2A subunit A protein gene expression in transgenic plants. The mutated gene will interact with the target wild-type gene in such a way that homologous recombination and targeted replacement of the wild-type gene will occur in transgenic plant cells, resulting in suppression of target protein activity.

Any of a number of genome editing proteins known to those of skill in the art can be used to mutate or knock out the target protein. The particular genome editing protein used is not critical, so long as it provides site-specific mutation of a desired nucleic acid sequence. Exemplary genome editing proteins include targeted nucleases such as engineered zinc finger nucleases (ZFNs), transcription-activator like effector nucleases (TALENs), and engineered meganucleases. In addition, systems which rely on an engineered guide RNA (a gRNA) to guide an endonuclease to a target cleavage site can be used. The most commonly used of these systems is the CRISPR/Cas system with an engineered guide RNA to guide the Cas-9 endonuclease to the target cleavage site.

CRISPR (Clustered Regularly Interspaced Short Palindromic Repeats)/Cas (CRISPR-associated) system, are adaptive defense systems in prokaryotic organisms that cleave foreign DNA. CRISPR loci in microbial hosts contain a combination of CRISPR-associated (Cas) genes as well as non-coding RNA elements which determine the specificity of the CRISPR-mediated nucleic acid cleavage. Three types (I-III) of CRISPR systems have been identified across a wide range of bacterial hosts. In the typical system, a Cas endonuclease (e.g., Cas9) is guided to a desired site in the genome using small RNAs that target sequence-specific single- or double-stranded DNA sequences. The CRISPR/Cas system has been used to induce site-specific mutations in plants (see Miao et al. 2013 Cell Research 23:1233-1236).

The basic CRISPR system uses two non-coding guide RNAs (crRNA and tracrRNA) which form a crRNA:tracrRNA complex that directs the nuclease to the target DNA via Wastson-Crick base-pairing between the crRNA and the target DNA. Thus, the guide RNAs can be modified to recognize any desired target DNA sequence. More recently, it has been shown that a Cas nuclease can be targeted to the target gene location with a chimeric single-guide RNA (sgRNA) that contains both the crRNA and tracRNA elements. It has been shown that Cas9 can be targeted to desired gene locations in a variety of organisms with a chimeric sgRNA (Cong et al. 2013 Science 339:819-23).

Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see Umov et al. 2010 Nat Rev Genet. 11(9):636-46.

Transcription activator like effectors (TALEs) are proteins secreted by certain species of Xanthomonas to modulate gene expression in host plants and to facilitate bacterial colonization and survival. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site have been found. The simple relationship between amino acid sequence and DNA recognition of the TALE binding domain allows for the design DNA binding domains of any desired specificity.

TALEs can be linked to a non-specific DNA cleavage domain to prepare genome editing proteins, referred to as TALENs. As in the case of ZFNs, a restriction endonuclease, such as FokI, can be conveniently used. For a description of the use of TALENs in plants, see Mahfouz et al. 2011 Proc Natl Acad Sci USA. 108:2623-8 and Mahfouz 2011 GM Crops. 2:99-103.

Meganucleases are endonucleases that have a recognition site of 12 to 40 base pairs. As a result, the recognition site occurs rarely in any given genome. By modifying the recognition sequence through protein engineering, the targeted sequence can be changed and the nuclease can be used to cleave a desired target sequence. (See Seligman, et al. 2002 Nucleic Acids Research 30: 3870-9 WO06097853, WO06097784, WO04067736, or US20070117128).

In addition to the methods described above, other methods for introducing genetic mutations into plant genes and selecting plants with desired traits are known. For instance, seeds or other plant material can be treated with a mutagenic chemical substance, according to standard techniques. Such chemical substances include, diethyl sulfate, ethylene imine, ethyl methanesulfonate (EMS) and N-nitroso-N-ethylurea. Alternatively, ionizing radiation from sources such as, X-rays or gamma rays can be used.

Also provided are methods of suppressing expression or activity of a polypeptide substantially identical to a protein of Table 1 or any one of SEQ ID NOS: 1-20 in a plant using expression cassettes that RNA molecules (or fragments thereof) that inhibit endogenous target expression or activity in a plant cell. Suppressing or silencing gene function refers generally to the suppression of levels of mRNA or protein expressed by the endogenous gene and/or the level of the protein functionality in a cell. The terms do not require specific mechanism and could include RNAi (e.g., short interfering RNA (siRNA) and microRNA (miRNA)), anti-sense, cosuppression, viral-suppression, hairpin suppression, stem-loop suppression, and the like.

A number of methods can be used to suppress or silence gene expression in a plant. The ability to suppress gene function in a variety of organisms, including plants, using double stranded RNA is well known. Expression cassettes encoding RNAi typically comprise a polynucleotide sequence at least substantially identical to the target gene linked to a complementary polynucleotide sequence. The sequence and its complement are often connected through a linker sequence that allows the transcribed RNA molecule to fold over such that the two sequences hybridize to each other.

RNAi (e.g., siRNA, miRNA) appears to function by base-pairing to complementary RNA or DNA target sequences. When bound to RNA, the inhibitory RNA molecules trigger either RNA cleavage or translational inhibition of the target sequence. When bound to DNA target sequences, it is thought that inhibitory RNAs can mediate DNA methylation of the target sequence. The consequence of these events, regardless of the specific mechanism, is that gene expression is inhibited.

MicroRNAs (niRNAs) are noncoding RNAs of about 19 to about 24 nucleotides in length that are processed from longer precursor transcripts that form stable hairpin structures.

In addition, antisense technology can be conveniently used. To accomplish this, a nucleic acid segment at least substantially identical to the desired gene is cloned and operably linked to a promoter such that the antisense strand of RNA will be transcribed. The expression cassette is then transformed into a plant and the antisense strand of RNA is produced. In plant cells, it has been suggested that antisense RNA inhibits gene expression by preventing the accumulation of mRNA which encodes the protein of interest.

Another method of suppression is sense suppression. Introduction of expression cassettes in which a nucleic acid is configured in the sense orientation with respect to the promoter has been shown to be an effective means by which to block the transcription of target genes.

For these techniques, the introduced sequence in the expression cassette need not have absolute identity to the target gene. In addition, the sequence need not be full length, relative to either the primary transcription product or fully processed mRNA. One of skill in the art will also recognize that using these technologies families of genes can be suppressed with a transcript. For instance, if a transcript is designed to have a sequence that is conserved among a family of genes, then multiple members of a gene family can be suppressed. Conversely, if the goal is to only suppress one member of a homologous gene family, then the transcript should be targeted to sequences with the most variance between family members.

Gene expression can also be inactivated using recombinant DNA techniques by transforming plant cells with constructs comprising transposons or T-DNA sequences. Mutants prepared by these methods are identified according to standard techniques. For instance, mutants can be detected by PCR or by detecting the presence or absence of PP2A subunit A mRNA, e.g., by northern blots or reverse transcription PCR (RT-PCR).

Catalytic RNA molecules or ribozymes can also be used to inhibit expression of embryo-specific genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs. The design and use of target RNA-specific ribozymes is well known.

The recombinant construct encoding a genome editing protein or a nucleic acid that suppresses expression may be introduced into the plant cell using standard genetic engineering techniques, well known to those of skill in the art. In the typical embodiment, recombinant expression cassettes can be prepared according to well-known techniques. In the case of CRISPR/Cas nuclease, the expression cassette may transcribe the guide RNA, as well.

In some embodiments, the genome editing protein itself, is introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell.

In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

Plant expression cassettes (e.g., for expression of the proteins described herein, or alternatively for expression of siRNA or gene editing proteins) can contain the polynucleotide operably linked to a promoter (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A number of promoters can be used. A plant promoter fragment can be employed which will direct expression of the desired polynucleotide in all tissues of a plant. In some embodiments, promoters described herein comprise 2 kb region upstream (5′) from where gene transcription is initiated.

Such promoters are referred to herein as “constitutive” promoters and are active under most environmental conditions and state of development or cell differentiation. Examples of constitutive promoters include the cauliflower mosaic virus (CaMV) 35S transcription initiation region.

Alternatively, the plant promoter can direct expression of the polynucleotide under environmental control. Such promoters are referred to here as “inducible” promoters. Environmental conditions that may affect transcription by inducible promoters include biotic stress, abiotic stress, saline stress, drought stress, pathogen attack, anaerobic conditions, cold stress, heat stress, hypoxia stress, or the presence of light.

In addition, chemically inducible promoters can be used. Examples include those that are induced by benzyl sulfonamide, tetracycline, abscisic acid, dexamethasone, ethanol or cyclohexenol.

Examples of promoters under developmental control include promoters that initiate transcription only, or preferentially, in certain tissues such as leaves, roots, fruit, seeds, or flowers. These promoters are sometimes called tissue-preferred promoters. The operation of a promoter may also vary depending on its location in the genome. Thus, a developmentally regulated promoter may become fully or partially constitutive in certain locations. A developmentally regulated promoter can also be modified, if necessary, for weak expression.

In some embodiments, the promoter directs expression in the exodermis, endodermis, phellem, or a sub-combination or combination of these. These are internal root tissues of the plant. Enhancing suberin in one or more of these tissues, can in some embodiments enhance tolerance to stresses and pathogens. Additionally, expression of suberin-enhancing proteins under the control of a phellem promoter can be used to improve tuber quality.

In some embodiments, the promoter directs expression in fruit epidermis. Such promoters can be used for expressing suberin-promoting genes in the epidermis of fruits to fortify the cuticule and reduce water loss, increase resistance to rotting, and increase shelf life of fruits.

Additional exemplary promoters include but are not limited to the following:

Exemplary Exodermis-Enriched Promoters:

Solyc12g005785, Solyc08g066890, Solyc07g049460, Solyc08g081555, Solyc09g082530, Solyc04g081860, Solyc07g052530, Solyc00g095860, Solyc08g078920, Solyc07g052540, Solyc10g076240, Solyc01g111230, Solyc08g075830, Solyc09g065430, Solyc12g006110, Solyc00g072400, Solyc10g076243, Solyc09g074890, Solyc07g047740, Solyc06g064960, Solyc05g012580, Solyc10g037880, Solyc12g096620, Solyc11g072110, Solyc08g066930, Solyc01g081250, Solyc03g005760, Solyc02g084850, Solyc10g007930, Solyc05g046020, Solyc12g049680, Solyc02g070080, Solyc06g060620, Solyc08g081780, Solyc12g011030, Solyc09g075670, Solyc11g012360, Solyc07g049240, Solyc10g009150, Solyc03g096420, Solyc08g074682, Solyc05g007470, Solyc12g097080, Solyc06g011350, Solyc08g014000, Solyc08g068780, Solyc09g082270, Solyc06g067870, Solyc08g061970, Solyc11g066270, Solyc08g079190, Solyc07g055060, Solyc02g092670, Solyc03g115690, Solyc09g007770, Solyc10g085880, Solyc03g120475, Solyc02g065780, Solyc08g066880, Solyc01g090610, Solyc01g066910, Solyc01g108860, Solyc10g083460, Solyc11g031950, Solyc08g008050, Solyc04g007400, Solyc11g011190, Solyc02g080200, Solyc06g060760, Solyc04g077670, Solyc08g079200, Solyc06g066830, Solyc09g089830, Solyc04g007750, Solyc12g009650, Solyc09g072590, Solyc03g096030, Solyc06g073460, Solyc07g043130, Solyc02g089250, Solyc09g098620, Solyc09g007760, Solyc01g109500, Solyc11g013810, Solyc06g060070, Solyc08g005960, Solyc06g075360, Solyc08g081190, Solyc01g096420, Solyc06g075650, Solyc12g005940, Solyc09g008320, Solyc12g056800, Solyc12g013690, Solyc02g086880, Solyc01g105410, Solyc09g014280, Solyc12g087940, Solyc03g111310, Solyc01g106780, Solyc01g097520, Solyc07g016215, Solyc02g080640, Solyc02g081400. Promoter designations are from Sol Genomics Network database, genome version Si 3.0.

Exemplary Fruit Epidermis-Enriched Promoters:

Solyc03g116100, Solyc05g053550, Solyc02g083860, Solyc11g013110, Solyc05g052240, Solyc09g091510, Solyc10g083440, Solyc02g089770, Solyc10g075090, Solyc01g079620, Solyc09g042670, Solyc06g060570, Solyc09g090980, Solyc09g092270, Solyc07g049440, Solyc10g075070, Solyc03g115220

Exemplary Phellem-Enriched Promoters:

Solyc12g036480, Solyc02g084790, Solyc06g009010, Solyc06g074390, Solyc01g090460, Solyc09g008250, Solyc11g072600, Solyc05g055480, Solyc09g008030, Solyc07g063420

Exemplary Endodermis-Enriched Promoters:

Solyc01g016460, Solyc01g067180, Solyc01g067230, Solyc01g067610, Solyc01g080580, Solyc01g081177, Solyc01g086893, Solyc01g090840, Solyc01g102450, Solyc01g108050, Solyc02g068645, Solyc02g083790, Solyc02g084260, Solyc02g085285, Solyc02g088517, Solyc02g088600, Solyc02g088983, Solyc03g046207, Solyc04g008780, Solyc04g051427, Solyc05g005877, Solyc05g013207, Solyc06g043260, Solyc06g043275, Solyc06g054600, Solyc06g072650, Solyc07g018144, Solyc08g061107, Solyc08g065820, Solyc09g010564, Solyc09g037087, Solyc09g037125, Solyc09g037130, Solyc09g065490, Solyc10g008620, Solyc10g044543, Solyc10g047643, Solyc10g074680, Solyc11g012563, Solyc11g027920, Solyc11g068630, Solyc12g005040, Solyc12g005130, Solyc12g006225, Solyc12g038350, Solyc12g042800, Solyc12g096270.

Exemplary Drought-Inducible Promoters:

Solyc06g076760, Solyc03g025810, Solyc12g010545, Solyc03g007230, Solyc12g006050, Solyc12g008430, Solyc02g086530, Solyc09g015070, Solyc12g089350, Solyc06g048860, Solyc06g068160, Solyc01g096320, Solyc11g071350, Solyc09g090790, Solyc09g082290, Solyc01g100090, Solyc02g090210, Solyc05g053160, Solyc01g060260, Solyc10g008700, Solyc01g006620, Solyc04g011600, Solyc03g006360, Solyc03g117800, Solyc11g067190, Solyc01g109920, Solyc09g097760, Solyc06g060970, Solyc08g067260, Solyc05g010330, Solyc03g112590, Solyc06g067980, Solyc10g078770, Solyc01g057000, Solyc08g078550, Solyc01g111040, Solyc12g009680, Solyc03g097585, Solyc01g087180, Solyc09g082550, Solyc01g103060, Solyc02g079640, Solyc07g055560, Solyc02g072540, Solyc11g009100, Solyc11g066700, Solyc08g079270, Solyc12g098900, Solyc06g076800, Solyc09g082340, Solyc06g060970, Solyc09g082280, Solyc03g097620, Solyc03g019820, Solyc01g099880, Solyc01g095320, Solyc09g015070, Solyc03g025810, Solyc06g051860, Solyc03g006360, Solyc11g007807, Solyc12g006050, Solyc12g098900, Solyc02g084850, Solyc02g061800, Solyc09g090800, Solyc10g079150, Solyc01g109920, Solyc03g044600, Solyc03g065250, Solyc08g081740, Solyc10g083690, Solyc03g097600, Solyc06g069070, Solyc04g071770, Solyc01g095305, Solyc01g096320, Solyc08g062960, Solyc03g095650, Solyc09g082300, Solyc03g007790, Solyc03g096670, Solyc08g078757, Solyc03g007230, Solyc03g013440, Solyc06g050800, Solyc08g075150, Solyc10g008700, Solyc04g016430, Solyc04g007470, Solyc10g024490, Solyc06g076400, Solyc09g083050, Solyc01g109810, Solyc01g057000, Solyc06g008580, Solyc08g068150, Solyc09g005610, Solyc12g010545, Solyc04g072700.

Solyc06g009370 is a robust meristematic cortex enriched promoter, stress independent in lateral roots (independent from drought and waterlogging in meristematic cortex and mature cortex).

Solyc08g081150 is a robust meristematic cortex enriched promoter, stress independent in lateral roots (independent from drought and waterlogging in meristematic cortex and mature cortex.

Additional Exemplary Endodermis Enriched Promoters:

Solyc01g016460, Solyc01g067180, Solyc01g067230, Solyc01g067610, Solyc01g080580, Solyc01g081177, Solyc01g086893, Solyc01g090840, Solyc01g102450, Solyc01g108050, Solyc02g068645, Solyc02g083790, Solyc02g084260, Solyc02g085285, Solyc02g088517, Solyc02g088600, Solyc02g088983, Solyc03g046207, Solyc04g008780, Solyc04g051427, Solyc05g005877, Solyc05g013207, Solyc06g043260, Solyc06g043275, Solyc06g054600, Solyc06g072650, Solyc07g018144, Solyc08g061107, Solyc08g065820, Solyc09g010564, Solyc09g037087, Solyc09g037125, Solyc09g037130, Solyc09g065490, Solyc10g008620, Solyc10g044543, Solyc10g047643, Solyc10g074680, Solyc11g012563, Solyc11g027920, Solyc11g068630, Solyc12g005040, Solyc12g005130, Solyc12g006225, Solyc12g038350, Solyc12g042800, Solyc12g096270

Methods for transformation of plant cells are well known in the art, and the selection of the most appropriate transformation technique for a particular embodiment of the invention may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens or Rhizobium rhizogenes-mediated transformation. Transformation means introducing a nucleotide sequence in a plant in a manner to cause stable or transient expression of the sequence.

In some embodiments, in planta transformation techniques (e.g., vacuum-infiltration, floral spraying or floral dip procedures) are used to introduce the expression cassettes of the invention (typically in an Agrobacterium vector) into meristematic or germline cells of a whole plant. Such methods provide a simple and reliable method of obtaining transformants at high efficiency while avoiding the use of tissue culture. (see, e.g., Bechtold et al. 1993 C. R. Acad. Sci. 316:1194-1199; Chung et al. 2000 Transgenic Res. 9:471-476; Clough et al. 1998 Plant J. 16:735-743; and Desfeux et al. 2000 Plant Physiol 123:895-904). In these embodiments, seed produced by the plant comprise the expression cassettes encoding the proteins. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown, and crossed with the same or different plant varieties using traditional breeding techniques to produce seed, which are then selected under the appropriate conditions.

An expression cassette can be integrated into the genome of the plant cells, in which case subsequent generations will express the encoded proteins. Alternatively, the expression cassette is not integrated into the genome of the plants cell, in which case the encoded protein is transiently expressed in the transformed cells and is not expressed in subsequent generations.

Any plant can be modified as described herein to have modulated amounts of suberin. Exemplary plants include species from the genera Arachis, Asparagus, Atropa, Aven, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and Zea. In some embodiments, the plant is a solanaceous plant. Exemplary solanaceous plants include but are not limited to tomato, potato, eggplant, and pepper.

### EXAMPLES

### Example 1

Description of Tomato Root Cell-Type Marker Lines and Translatome Experiment

We conducted translatome profiling of 1 cm of the tomato root tip to obtain cell type resolution translatome patterns (transcripts associated with ribosomes). We used twelve TRAP marker lines marking the following cell types: the epidermis (AtWERpro) promoter, distinct populations of the cortex including cells within the meristem (SlCO2pro), all cortex cell layers and developmental stages (SlPEPpro), and the non-exodermal cortex (AtPEPpro), the endodermis (SlSCRpro), the quiescent center (SlWOXSpro and SlSCRpro), xylem cells (AtS18pro), phloem cells (AtS32pro), all vascular cells (SlSHRpro), all cells within the root meristem (SlRPL11Cpro) and two constitutive promoters (35Spro and SlACT2pro) (promoter details as in Ron M. et al. (2014), Plant Physiology 166: 455-469. The plant growth and TRAP-seq protocols are as described before (Reynoso M A et al. (2019), Science 365: 1291-1295). Phylogenetic and cell type-resolution data identified novel genes associated with exodermal suberin biosynthesis. Tomato candidate genes were identified via integrated phylogenetic and tomato cell type TRAP-seq. Exodermal suberin deposition was reduced in a CRISPR-Cas9 mutant allele, visualized by Fluorol Yellow staining and quantified. Dynamic expression of rice GPATs during drought; candidate ortholog was determined. Statistically significant. differences were determined using one-way ANOVA. Species that were included in the phylogenetic tree: A. thaliana (At), tomato (Sl), rice (Os), tobacco (Nt), maize (Zm), apple (Md), M. truncatula (Mt), soybean (Gm), grape vine (Vv), sorghum (Sb), B. distachyon (Bd), cork oak (Qs), and S. moellendorffi (Sm).

Data Analysis

To identify genes with enriched expression within each cell type, we employed two independent approaches: the Brady method (Brady S. M. et al. (2007), Science, 318, 801-806) and the modified ROKU method (Song et al., Developmental Cell 2016). Briefly, the Brady method is based on the intersection of differentially expressed genes (log₂ FC≥2 and FDR≤0.05) within all pairwise comparisons of non-overlapping cell types. The modified ROKU method calculates entropy for each gene and determines the outlier cell type. A union gene set of the two approaches was created for each cell type, and a non-redundant list of enriched genes was curated by including only genes with a TPM value≥2 that have the highest expression in the target cell type compared with all other cell types, excluding the two constitutive promoters.

Hairy Root Transformation+Function

To design the sgRNAs (guide RNAs) we used two online platforms: The CRISPR-PLANT and CRISPR-P. The CRISPR cloning was made using a modified protocol from Lowder et al., Plant Physiology (2015) and Ron M. et al. (2014), Plant Physiology 166: 455-469. To generate the mutants, we took advantage of the hairy root transgenic system (Ron M. et al. (2014), Plant Physiology 166: 455-469). For example, a pipeline to mutate selected candidate genes and phenotype roots follows: 1. sgRNAs are cloned in a common vector with the Cas9 gene. Rhizobium (Agrobacterium) rhizogenes is used to infect cotyledons. 2. Transgenic hairy roots are formed. Each root represents a single transformation event. 3. 10-15 roots are genotyped and homozygous mutant lines selected. 4. These are phenotyped along with non-transgenic controls for suberin deposition.

Moreover, to increase the efficiency of the CRISPR genome edits by using heat stress we also adapted the protocol from LeBlanc et al, Plant Journal (2018) to be applied in the hairy root system. We have been able to rapidly screen 39 CRISPR mutants for transcription factors enriched in the exodermis and 4 CRISPR mutants for suberin biosynthesis enzymes in over a year. The mutants were phenotyped for suberin deposition in the root using histochemical analyses to detect suberin with the Fluorol yellow dye (Naseer S. et al., Proc Nal Acad Sci USA., 2012 Jun. 19; 109(25):10101-6. doi: 10.1073/pnas.1205726109. Epub 2012 Jun. 4).

Phylogenomic Approach

Using cell type-specific gene expression for tomato, arabidopsis and rice in combination with phylogenomics analyses, we identified a set of novel tomato genes involved in suberin biosynthesis. Here, we provide an example of how we used this platform to functionally predict likely GPAT (Glycerol-3-phosphate Sn-2-acyltransferase) enzymes in tomato. GPAT members participate in polyester biosynthesis, but one of our two potential candidates (i.e. GPAT4) was previously described to participate in cutin formation in the Arabidopsis shoot with no known role in suberin formation (Beisson et al., Current Opinion in Plant Biology 20121. GPAT members participate in polyester biosynthesis, but one of our two potential candidates (i.e. GPAT4) was previously described to participate in cutin (A suberin-like polymer) formation in the Arabidopsis shoot with no known role in suberin formation (Beisson et al., Current Opinion in Plant Biology 2012). Using the hairy root system (Ron M. et al. (2014), Plant Physiology 166: 455-469), CRISPR-mediated deletion alleles were rapidly generated and phenotyped using histochemical analyses. The data confirm the requirement of GPAT4 for suberin production.

Data Indicating that these Genes are Necessary for Suberin Biosynthesis.

Hairy Root Data

We have analyzed the CRISPR mutants for suberin deposition using histochemical analyses to detect suberin using Fluorol Yellow. Suberin quantification was performed after fluorol yellow staining of selected mutant lines for both suberin biosynthetic genes and transcription factors.

Effects of Candidate Gene Knock Out on Suberin Composition

We performed mass spectrometry on root suberin samples to quantify the components of suberin from some of our validated candidates based on the previous histological analyses. Every single mutated gene was validated in this second analysis for altered suberin composition and indicates that the fluorol yellow quantification approach is sufficient to determine perturbed suberin levels, and thus all these genes are suberin biosynthetic enzymes

TABLE 1
D. List of Genes

| | | |
|---|---|---|
| NAME | MUTANT CODE | GENE ID |

TRANSCRIPTION REGULATORS

| | | |
|---|---|---|
| SlMYB41 | myb02 | Solyc02g079280 |
| SlMYB74 | myb j | Solyc10g005460 |
| SlMYB92 | myb f/myb05 | Solyc05g051550 |
| SlMYB63 | myb l/myb15 | Solyc10g005550 |
| SlMYB106 | myb c | Solyc02g088190 |
| SlMYB52 | myb d | Solyc03g093890 |
| SlMYB37 | myb i | Solyc09g008250 |
| SlBLH2 | HBT6 | Solyc06g074120 |
| SlJMJ11 | lsd | Solyc04g028580 |
| SlEBP2b | ebp | Solyc08g082210 |
| SlHLH069 | bhlh | Solyc11g010340 |
| SlC2H2 | c2h2 a | Solyc01g099340 |

BIOSYNTHETIC GENES

| | | |
|---|---|---|
| SlASFT | asft | Solyc03g097500 |
| SlGPAT5 | gpat a/gpat4 | Solyc04g011600 |
| SlGPAT4 | gpat b/gpat5 | Solyc01g094700 |
| SlLACS4 | lacs | Solyc01g095750 |
| SlCYP86A | cyp a | Solyc01g094750 |
| SlFAR3A | far a | Solyc06g074390 |
| SlFAR3B | far b | Solyc11g067190 |
| SlKCS2 | kcs | Solyc09g083050 |

### Example 2

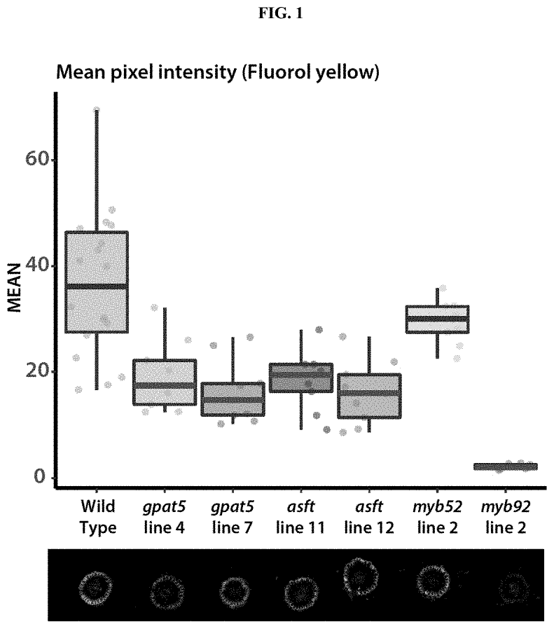

Prior data was generated from CRISPR-Cas9 edited roots generated by Rhizobium rhizogenes (the microbe used for transformation). Transgenic plants with biallelic, sequence confirmed deletions in the genes below were generated using Agrobacterium tumefaciens. Thus whole plants were generated where the edited genes are passed on through sexual reproduction, via the gametes. As shown in FIG. 1, it was confirmed that reduction of suberin occurs in plants carrying GPAT5, ASFT and MYB92 deletion alleles.

Although the foregoing invention has been described in some detail by way of illustration and example for purposes of clarity of understanding, one of skill in the art will appreciate that certain changes and modifications may be practiced within the scope of the appended claims. All patents, patent applications, and other publications, including GenBank Accession Numbers, cited in this application are incorporated by reference in the entirety for all purposes.

Exemplary Sequences (Obtained from the Sol Genomics Network Database, Version ITAG 3.2):

>Solyc02g079280.3.1
SEQ ID NO: 1:
MGRAPCCDKNGLKKGPWTPEEDQKLIDYIQKHGYGNWRTLPKNAGLQRCGKSCRLRWTNY
LRPDIKRGRFSFEEEETIIQLHSILGNKWSAIAARLPGRTDNEIKNYWNTHIRKRLLRMG
IDPVTHSPRLDLLDLSSILNHSIYNNSSHHQMNLSRLLGHVQPLVNPELLRLATSLISSQ
RQNTNNFLIPNNLQENQIICQNQLPQMVQNNQIQDFSTISTTPCVPFSSHEAQLMQPPIT
TKIEDFSSDLENFGNSQNNCQVINDDEWQLSNGVTDDYFPLQNYGYYDPLTSDENNNNFN
LQSVVLSNLSTPSSSPTPLNSNSTYFNNSSSTTTEDERDSYCSNMLNFDNIPNIWDTTNE
FM*
>Solyc10g005460.3.1
SEQ ID NO: 2:
MGRTPCCDKNGLKKGPWTTEEDQKLIDYIQKYGSGNWRILPKNAGLQRCGKSCRLRWINY
LRPDIKRGKFSFEEEETIIHLHSILGNKWSAIAARLPGRTDNEIKNYWNTNIRKKLLRMG
IDPITHSPRLDLLDLNSIFNPSLYNSTQLDNNISRLLGVQSLVNPEILRLANSLLSSHHQ
NQNFLLQSNFQENQLCNSYVQNKLTPFGQTSLIQNPINNISTCSNFNTPSVPFYSDTLAM
QQPNVEEQSSSNILNFNSQNFTFNSILPTLSTPSSTPTSLNSNSSTISEEERESYCSMLN
FDIPNILDVNEFM*
>Solyc05g051550.2.1
SEQ ID NO: 3:
MGRSPCCDENGLKKGPWTPEEDQKLTNHINKHGHGSWRALPKLAGLNRCGKSCRLRWSNY
LRPDIKRGKFSQEEEQTILNLHAVLGNKWSAIATHLPGRTDNEIKNFWNTHLKKKLIQMG
YDPMTHRPRTDIFDSLQHLIALVNLKELIESHSWEEQAMRLHYLQNLLQQPHNNMSTLSG
IQNVEAYNLLNSLGDSQFLSTNNNNLGNHIVQQIPSSLDQPIIQDSISFSHLPELHTPSS
FQTSLNKDRVRTEDTEFRIMSQGETSPASPWLPSLSPPPPPQVMNDQRSKENSSEVVISS
GLSGESKNSNHLFLPDNKQSLNNIEEAPPSIWSDLLEDSFFQDIDKF*
>Solyc10g005550.2.1
SEQ ID NO: 4:
MGKGRTACCDKSKVKKGPWTPSEDLKLISFIQKHGHGNWRALPKQAGLLRCGKSCRLRWI
NYLRPDVKRGNETPQEEDTIINLHRAFGNRWSKIASHLPGRTDNEIKNVWNTHLKKRLVV
MKKEECKSSSSSTSTSSHQGQYMDNNNNNNSNTLESFSPTSSKANDQVMDFWEYMLDTSS
TTTSINNLDHLDSYSKLDITSEHHPQQLVDEYECQKWLTYLEIELGLTTNNQQEDHQNNF
MQL*
>Solyc02g088190.3.1
SEQ ID NO: 5:
MGRSPCCDKVGLKKGPWTPEEDQKLLAYIEEHGHGSWRALPTKAGLQRCGKSCRLRWTNY
LRPDIKRGKFTLQEEQTIIQLHALLGNRWSAIATHLSKRTDNEIKNYWNTHLKKRLVKMG
IDPVTHKPKNDALLSNDGQSKNAANLSHMAQWESARLEAEARLARQSKLRSNSFQNSLAS
QEFTAPSPSSPLSKPVVAPARCLNVLKAWNGVWTKPMNEGSVASASAGISVAGALARDLE
SPTSTLGYFENAQHITSSGIGGSSNTVLYEFVGNSSGSSEGGIMNNDESEEDWKEFGNSS
TGHLPQYSKDVINENSISFTSGLQDLTLPMDTTWTTESSRSNTEQISPANFVETFTDLLL
SNSGDGDLSEGGGTESDNGGEGSGSGNPNENSEDNKNYWNSIFNLVNNPSPSDSSMF*
>Solyc03g093890.3.1
SEQ ID NO: 6:
MPRVQQQQQKGTSMEAIIKKGAWSPEEDQKLRGYIMKYGIWNWRQMPKFAGLSRTGKSCR
LRWMNYLRPDVKRGPFTTEEVEIVIKTYQELGNSWSAIAAKLPGRTDNEVKNFFHTHLKK
HLGLKNHDVPLKTRKIRKQTKEDEKKISTRGRLVLETSNNSNLLTTDVCSPCSSITTCEE
NQMMDPFVNFSQTFEVCYNNITSLVVDQQVPGMEHTCINIGVAQPHSIPHGPAVNSFDQF
DMNSFWIDVLGNI*
>Solyc09g008250.3.1 blind-like1
SEQ ID NO: 7:
FSKISSQEKKIIIIIIIMGRAPCCDKANVKKGPWSPEEDAKLKEYIDKFGTGGNWIALPQ
KAGLRRCGKSCRLRWLNYLRPNIKHGEFSDEEDRIICSLYANIGSRWSIIAAQLPGRTDN
DIKNYWNTKLKKKLMGFVSSSHKIRPLNHHDYHHQIPTNCYNNYSSLVQASSLLISSNYP
NNTTFPCYETNIPSTTPSSTSFLSAGASTSCTSGITASTFAGRTTSSDESYDISNFNFHS
YMYNNNGVISEGEKLISGNNASGCYVDEQQNPLDYSSLEEIKDLISTNHGTCNSTSFLLD
HEIKTEEKVIMYY*
>Solyc06g074120.3.1
SEQ ID NO: 8:
MYYQGTSDNNIQADHHQQQHNNLGNSNNNIQTLYLMNPNSYMQGYTTTDTQQHLQQQQNQ
HQLLFLNSAPAGGNALSHANIQHAPLQQQHFVGVPLPAVSLHDQINHHGLLQRMWNNQDQ
SQQVIVPSSTVVSATSCGGTTTDLASQLAFQRPIVVSPTPQHRQQQQQQGGLSLSLSPQQ
QQQ1SFNNNISSSSPRTNNVTIRGTMDGCSSNMILGSKYLKAAQELLDEVVNIVGKSNKG
DDQKKDNSMNKELIPLVSDVNTNSSGGGGGESSSRQKNEVAIELTTAQRQELQMKKAKLL
AMLEEVEQRYRQYHHQMQIIVSSFEQVAGVGSAKSYTQLALHAISKQFRCLKDAISEQVK
ATSKSLGEDEGLGGKIEGSRLKFVDHHLRQQRALQQLGMMQPNAWRPQRGLPERAVSVLR
AWLFEHFLHPYPKDSDKIMLAKQTGLTRSQVSNWFINARVRLWKPMVEEMYLEEVKNQEQ
NSSNTSGDNKNKETNISAPNEEKQPIITSSLLQDGTTQAEISTSTISTSPTAGASLHHAH
NFSFLGSFNMENTTTTVDHIENNAKKPRNHDMHKFSPSSILSSVEMEAKARESTNKGFTN
PLMAAYAMGDFGREDPHDQQMTANEHGNNGVSLTLGLPPSENLAMPVSQQNYLSNELGSR
PEIGSHYNRMGYENIDFQSGNKRFPTQLLPDFVTGNLGT*
>Solyc04g028580.2.1
SEQ ID NO: 9:
MDDIPEWLKGLPLAPEFRPTDTEFADPIAYISKIEKEASAFGICKVIPPLPKPSKKYVLH
NLNNSLSKCPDLNSAGAPVFTTRHQELGHTEKKKFPFGAQKQVWQSGQLYTLDQFETKSK
NFARTQFGIVKDISPFLVEAMFWKTAFDHPIYVEYANDVPGSAFGEPEENFCRTKRPRNR
KILDRTSSTTSVDKGRSHHSVDTPSSSLLTPLSNSSPFRPKGCSNAAEMEGSAGWKLANS
PWNLQVIARSPGSLTRFMPDDIPGVTSPMVYIGMLFSWFAWHVEDHELHSLNFLHTGSPK
TWYAVPGDYAFSFEEVIRCHAYGETTDRLVNLGHKALKFASGKAKATYTEQHEDFVVRLC
TSNHEIGCLGEQAALALLGEKTTLLSPEVLVASGIPCCRLVQNPGEFVVTFPRAYHVGFS
HETEIFIGLRTYDSLWRS*
>Solyc08g082210.3.1
SEQ ID NO: 10:
MSNSPVFEPLGTSVYLRQRDLLQKFCQENIANISIPTTSKTIPFRNSLYTQSYKLPEKKK
LYRGVRQRHWGKWVAEIRLPQNRMRVWLGTYETAEAAAYAYDRAAYKLRGEYARLNFPNV
RDPSKLGFGDGEKMNAVKNAVDAKIQAICQRVKREKAKKAAKKKSENENGLWRSEDSTCS
VFGDCLKDPLMESEFDSCSLARMPSFDPELIWEVLAN*
>Solyc11g010340.2.1
SEQ ID NO: 11:
MALETVVFQQDPFNYSHKDCNFYNLETFHDYGNFGYEGYNWNSSIPQSYNDDDNNNNINN
NNSNSSPDKYFPVESTVVSGRRKRRRTKCAKNEEEIHNQRMTHIAVERNRRRQMNDYLAV
LRSLMPPSYAQRGDQASIVGGAINFVKELEQLLQFLEAHKQVITTNQQHIQYSSFSKFFT
FPQYSTGNNNHPLAATTSNEGSEERRSAVADIEVTMVESHANVKVLSRRRPKQLLKIVNW
LQAMCLTILHLSVTTADHMVLYTFSVKVEENCELNTVSEIASAVHEMVAMIKEEAMPC*
>Solyc01g099340.3.1
SEQ ID NO: 12:
MSNSNLSSGNSSEEADETPYVLSSTSDGSSAHQQHSQTNNKKRRKLPGNPDPSAEVIALS
PKTLMATNRFICEVCNKGFQREQNLQLHRRGHNLPWKLKQKTSNEIKKRVYICPESSCIH
HNPSRALGDLTGIKKHFSRKHGEKKWKCEKCSKKYAVQSDWKAHSKTCGTKEYKCDCGTI
FSRRDSFVTHRAFCDALAEENNKVNQVLASTTQPLATGPELISTTQMLNLPQIRNSNMKI
PSIPLNMAGSMFSSSSGFNQLGTNSSNMSSATALLQQAAQMGATVNNNMNSTLFNGVQIP
IQSNHDHDQNETQIGSILQGFGGSMLQNNGDDHHKSSRVLQNEQGWFNNNNNNSNTGLFN
EKQRTLNKEAGHSNEESLTLDFLGIGGMRHRNLHEMHQHQQEMSFEQQQVNHQSIQRVNS
IWDD*
>Solyc03g097500.3.1
SEQ ID NO: 13:
MENGKHSVAIELTVKQGVPSLVSPAEETEKGPYYLSNLDQNIAVPVRTIYCFKSEEKGND
NAAEVMKDALSKVLVHYFPLAGRLTISQEMKLIVDCSGEGAVFVEAEANCNIEDIGDNTK
PDPVTLGKLVYDIPGAKNILEMPPLVAQVTKFKCGGFVLGLCMNHCMFDGIGAMEFVNSW
GEIARGLPIKVPPFLDRSILKPRNPPKPEYTHNEFAEIKDISDSTKLYQEEMMYKAFCFD
PEKLEQLKAKAKEDGNVTKCTSFEVLSAFIWKARTQALQMKPDQKTKLLFAVDGRSREDP
SIPRGYFGNGIVLTNALCTAAEIVENPLSVAVKLVQEAVKLVTDSYMKSAIDYFETTRAR
PSLTATLLITTWSRLSFHTTDFGWGEPIVSGPVALPEKEVSLFLSHGKERRSVNVLLGLP
ASAMKTFEELMEI*
>Solyc04g011600.3.1
SEQ ID NO: 14:
MDSSIVCELEGTLLKDQDPFSYFMLIAFEASSLIRFAILLMLWPLIKFLGICGQKDKGLK
LMIFVATIGVKISEIEVVARAVLPKFYFDDIDMKSWRIFSSFDKRIVVTKIPRIMVERFV
KEHLRADDVIGSELVVNNFGFATGFIKDDFDSILERVGALFDGETQPSLGLGRPQNGSSF
LSLCKEQLHPPFMINKNQDHIIKPLPVIFHDGRLVKRPTPSIALLILLWIPFGIILATIR
IIIGLILPLWIVPYLAPLFGGKVIVKGKPPPPASITNSGVLFVCTHRTLLDPVVLSTVLQ
RRIPAVTYSISRLSEILSPIPTVRLTRIREVDAQKIKRQLEKGDLVVCPEGTTCREPFLL
RFSALFAELTDRIVPVAMNYRVGFFHATTARGWKGMDPIFFFMNPRPMYEVTFLNQLPVE
ATCSSGKSPHDVANYVQRILAATLGFECTNFTRKDKYRVLAGNDGIVSQNSGTNLANKFK
KWATFKLFIH*
>Solyc01g094700.3.1
SEQ ID NO: 15:
MSLPKSKKSFPSVTTCDTSAVNHHSVAADLDGTLLISRSSFPYFMLVAIEAGSLFRGLIL
LLSFPLIAIAYVFVSEALAIQMLIYISFAGLKVRDIELASRAVLPRFYATDVRKESFEVF
DQCKRKVVVTANPTIMVEPFVKDFLGGDKVLGTEIEVNPKTKKATGFVKSPGILVGKWKK
LSILKEFGEEMPDIGLGDRESDHDFMSICKEGYMVLPSESAKPVPLDRLKSRLIFHDGRL
VQRPTPFNALVTYIWLPFGFALGVFRVYFNLPLPERIVRYTYGMVGINLVIKGPRPPPPS
PGTPGNLYVCNHRSALDPIVIAIALGRKVSTVTYSVSKLSRFLSPIPAIALTRDREADAA
MIKKLLEKGDLVVCPEGTTCREPFLLRFSALFAELSDRIVPVAVDTKQSMFFGTTVRGVK
FWDPYFFFMNPRPTYELTFLEPLPMEMTCKAGKTSIEVANHVQKVLGGVLGFECTQLTRK
DKYMLLGGNDGKVESMYSKKA*
>Solyc01g095750.2.1
SEQ ID NO: 16:
MEDQKKLYVFEVEKAKEVRSNGRPSRGPVYRNVLAKDGFRPLSQSLQSCWDIFCESVRKF
PHNRMLGEREMSHGQAGKYIWLTYREVYDLVLKVGASMRVCGVKQVRLSYCKIKDIQGGK
CGIYGANCSNWVISMQACNALGLYCVPLYDTLGAGAVEYIICHAEVSVAFAEETKIFEVL
KAFPNAGKFLKSLISFGKVTQEQKDMAGNFDLKLYSWDEFLLLGMQEKFDLPAKKKTDIC
TIMYTSGTTGDPKGVMISNESILSLISGVNHHMETVGEEFTDKDVYLSYLPLAHIFDRVI
EELFISKGASVGFWHKDVKQLIDDIKELKPTVFCSVPRVLDKIYSGLVEKISCAGFLKHK
LFNFTYNYKLGNMSKGYRHSEAAPIFDKIIFNKVKEGLGGNLRLILSGAAPLSSTVETYL
RVVTCANVLQGYGLTETCAGSFVARPDELAMVGTVGPPLPIIDVCLESVPEMGYDALGDT
PRGEICIRGKCLFSGYYKREDLTKEVLVDGWFHTGDVGEWQPDGSMKIVDRKKNIFKLSQ
GEYVAVENLEGIYSLASSVDSIWIYGSSYESFLVAVVNPNMEALRSWANENGMTGDFDTI
CENPKAKAYILSELTNIAKEKKLKGFEFIKAVHLDPVPFDMERELITPTHKKKRAQFLKY
YQNNIDTLYKNTR*
>Solyc01g094750.3.1
SEQ ID NO: 17:
MDIAIALLLFSFITCYLLWFTFISRSLKGPRVWPLLGSLPGLIENSERMHEWIVDNLRAC
GGTYQTCICAIPFLARKQGLVTVTCDPKNLEHILKTRFENYPKGPTWQAVFHELLGQGIF
NSDGDTWLFQRKTAALEFTTRTLRQAMARWVNRAIQLRFCPILKTAQVEGKPVDLQDLLL
RLTFDNICGLAFGKDPQTLAPGLPDNTFASAFDRATEASLQRFILPEVVWKLKKWLGLGM
EVSLNRSLVQLDKYMSDIINTRKLELMSQQKDGNPHDDLLSRFMKKKESYTDKFLQHVAL
NFILAGRDTSSVALSWFFWLVIQNPVVEQKILQEISTVLVETRGSDTSSWLEEPLAFEEV
DRLTYLKAALSETLRLYPSVPEDSKHVVVDDVLPDGTFVPAGSSITYSIYSAGRMKSTWG
EDCLEFKPERWLTLDGKKFVMHEQYKFVAFNAGPRICLGKDLAYLQMKSVAAAVLLRHRL
TVAPGHKVEQKMSLTLFMKDGLKVNLRPRELTPFVNSVKEVQLIQI*
>Solyc06g074390.3.1
SEQ ID NO: 18:
MELTSVLKFLENRAILVTGATGFLAKIFVEKILRVQPNVKKLYLLLRAQDNNAALQRFNN
EAVAKDLFKLLREKHGANLNTFISERTTIIPGDITIENLGVKDTNLLEEMWREVDVVVNL
AATTNFDERYDVALGLNTFGAINVLNFAKKCSKLKVLLHVSTAYVSGEKRGLILETPYNL
GETLNGTSGLDIYTEKKVMEETLKQLRVEGSSQESITSAMKELGLQRARKYGWPNPYVFT
KALAEMILGDMKEDVLLVIFRPTIVTSTLRDPFPGWVEGIRTIDSLAVGYGKGKLTCFLG
DPEAIIDLIPADMVVNAMIVTMMAHADQRGSQIIYHVGTSVSNPVKFTCPQEYAFRHFKE
HPWIDKQGKPVIVGKVNVLSSMDSFRRYMALRYMLPLKGLEIVNTILCQFFQDKYSELDR
KIKFVMRLIDLYEPYLFFKGVYDDMNTEKLRRAAKESGIETDVFNFNPKSINWEDYFMNT
HIPGWKYVFK*
>Solyc11g067190.2.1
SEQ ID NO: 19:
MEMTSVLNFLENRTILVTGATGFLAKIFVEKILRVQPYVKKLYLLLRAADDKSAMQRFNT
EVVGKDLFKVLREKCGPNFTTFVSQRTTIVPGDITCENLGVNDTNLLEQMWKEVDIVVNL
AATTNFDERYDVALGLNTFGASHVLNFAKKCNKLKVLLHVSTAYVCGEKEGLMLEKPYYM
GETLNGTLGLDIEAEKKVMDEKLKQLKAENASEKSITTAMKELGLERARKYGWPNTYVFT
KAMGEMLLGKLKEEVPLVINRPTIITSTFKEPFPGWVEGIRTIDSLAVGYGKGRITCFLG
NPKTILDVIPADMVVNSMIVAMMAHADQKGSETIYQIGSSVSNPLNITNLRDYGFNYFRK
NPWINKVNGKPIIVGKVNVLSSMDSFQRYMALHYILPLKGLEIVNAAFCQYFQGKYLELY
KKIKFVMRLIDLYGPYLFLKAAFDDLNTEKLRIGAKESGIETEIFYFDPKIINWEDYFMK
IHLPGVVRYVFK*
>Solyc09g083050.3.1
SEQ ID NO: 20:
MGDESTRRVSIEANSNKLPNFLLSVRLKYVKLGYHYLISHAMYLFLIPILMALFAHLSTI
TMEDMVQLWNQLKFNLVTVILCSALIVFLATLYFMTRPRKVYLVDFSCYKPKPEVMCPKE
LFMERSKLAGIFTEENLAFQKKILERSGLGQKTYFPEALLKLPPNPCMAEARKEAEMVMF
GAIDELLEKTGVKAKDIGILVVNCSLFNPTPSLSAMIVNHYKLRGNILSYNLGGMGCSAG
LISIDLAKQMLQVQPNSYALVVSMENITLNWYFGNNRSMLVSNCIFRMGGAAILLSNKSS
DRKRSKYQLIHTVRTHKGADDKSYGCVFQEEDDNKKIGVALSKDLMAVAGEALKTNITTL
GPIVLPMSEQLLFFATLVARKVLKMKIKPYIPDFKLAFEHFCIHAGGRAVLDELEKNLEL
SEWHMEPSRMTLYRFGNTSSSSLWYELAYTEAKGRIKKGDRTWQIAFGSGFKCNSAVWCA
LRTINPAKEKNPWMDEIDEFPVEVPRVVTINDS*

1. A plant having increased suberin, wherein the plant ectopically expresses or overexpresses one or more polypeptide that is substantially identical to one or more protein as provided in Table 1 or SEQ ID NOS: 1-20, wherein the plant has increased suberin compared to a control plant not ectopically expressing or overexpressing the one or more polypeptide.

2. The plant of claim 1, wherein the plant is a Solanaceous plant.

3. The plant of claim 1, wherein the plant comprises an expression cassette comprising a promoter operably linked to a polynucleotide encoding one of the polypeptides of Table 1 or SEQ ID NOS: 1-20.

4. The plant of claim 3, wherein the promoter is inducible or tissue-specific.

5. A tuber from the plant of claim 1.

6. A method of making suberin, the method comprising,
providing the plant or tuber of claim 1; and
extracting suberin from the plant or a part of the plant.

7. A method of cultivating plants that are tolerant to drought or high salinity conditions, the method comprising,
cultivating the plant of claim 1 under high salinity or drought conditions.

8. A plant having decreased suberin, wherein the plant is (a) mutated to reduce or knockout expression, or (b) expresses an siRNA or antisense polynucleotide to reduce expression, of one or more polypeptide that is substantially identical to one or more protein as provided in Table 1 or SEQ ID NOS: 1-20, wherein the plant has decreased suberin compared to a control plant that expresses the one or more polypeptide.

9. The plant of claim 8, wherein the plant is a Solanaceous plant.
